(19) United States
(12) Patent Application Publication
(10) Pub. No.: US 2023/0058835 A1
(43) Pub. Date: Feb. 23, 2023

(54) SYSTEMS AND METHODS FOR MEDIA-BASED PERSONALITY ANALYSIS

(71) Applicant: Keeper Recruitment Corporation, New York, NY (US)

(72) Inventors: Vishal Ahluwalia, New York, NY (US); Dean Graziano, Mill Creek, WA (US); Derek Stanford, Bothell, WA (US); Gnana Natarajan, Dacula, GA (US); Victor H. Aviles, Bronxville, NY (US)

(21) Appl. No.: 17/891,022

(22) Filed: Aug. 18, 2022

Related U.S. Application Data
(60) Provisional application No. 63/234,605, filed on Aug. 18, 2021.

(51) Int. Cl. G06Q 10/06; G06Q 10/10; G06F 16/9535

(52) U.S. Cl. CPC G06Q 10/063118; G06Q 10/103; G06F 16/9535

(57) ABSTRACT

Systems and methods for providing personality factor determination for users, teams, and organizations, including determining personality traits and generating a personality factor score for each personality trait. The systems and methods give insight to personality traits of users teams and organizations through the use of personality profiles and by identifying correlations between personality traits of users, teams, and organizations. The systems and methods utilize web-based technology that includes one or more of media capture, video sampling, peer collaboration, and text-based descriptors. Media may serve as proxy measures or representations of personality traits. A personality profile may be generated for a user, team, or organization, which includes representations of their personality traits. The representations may be used to evaluate users, teams, and organizations based on their personality traits.

### BACKGROUND

### Technical Field

The present disclosure generally relates to computer-based systems and methods and, more particularly, to computer-based systems and methods for determining, analyzing, and using personality traits of people and groups to improve various systems and processes.

### Description of the Related Art

Creating a personality profile for an individual or group is currently difficult and impractical for a variety of reasons. One such reason is that the personality of a person or group is difficult to quantify, and typically requires data obtained from tests or professionals. Furthermore, obtaining a personality profile for a single person often incurs a large resource cost. As a result, it is not feasible, or efficient, for many organizations to obtain personality profiles for their members and for groups within the organization. Additionally, organizations typically do not have the resources to measure, analyze, or search for personality profiles of members or groups who wish to join the organization. Thus, there is a need to provide improved systems and methods for obtaining personality profiles of individuals and groups, such as employees and teams, in a way that eliminates the inefficiencies of current practices.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not necessarily drawn to scale, and some of these elements may be arbitrarily enlarged and positioned to improve drawing legibility. Further, the particular shapes of the elements as drawn, are not necessarily intended to convey any information regarding the actual shape of the particular elements, and may have been solely selected for ease of recognition in the drawings.

FIG. 1 is a diagram that illustrates an environment in which a personality factor determination system of the present disclosure may operate.

FIG. 2 shows an example attribute selection interface of the personality factor determination system, according to one illustrated implementation.

FIG. 3 is an example visual content selection interface, presented by the personality factor determination system in some embodiments.

FIG. 4A is an example personality score screen, presented by the personality factor determination system in some embodiments.

FIG. 4B is an example personality trait prediction screen, presented by the personality factor determination system in some embodiments.

FIG. 5A is an example user dashboard presented by the personality factor determination system in some embodiments.

FIG. 5B is an example personality factor graph screen, presented by the personality factor determination system in some embodiments.

FIG. 5C is an example values graph screen, presented by the personality factor determination system in some embodiments.

FIG. 5D is an example affinity group screen, presented by the personality factor determination system in some embodiments.

FIG. 5E is an example “mood gauger” and feed screen, presented by the personality factor determination system in some embodiments.

FIG. 5F is a first portion of an example insight report screen, presented by the personality factor determination system in some embodiments.

FIG. 5G is a second portion of an example insight report screen, presented by the personality factor determination system in some embodiments.

FIG. 5H is a first example personality integration screen, presented by the personality factor determination system in some embodiments.

FIG. 5I is a second example personality integration screen, presented by the personality factor determination system in some embodiments.

FIG. 5J is a third example personality integration screen, presented by the personality factor determination system in some embodiments.

FIG. 6 is an example activity screen, presented by the personality factor determination system in some embodiments.

FIG. 7 is a logical flow diagram generally showing one embodiment of a process for generating a personality factor score for a user, according to one non-limited illustrated implementation.

FIG. 8 is a logical flow diagram generally showing one embodiment of a process for identifying a user group and determining how a group personality profile changes based on membership of a particular user, according to one non-limited illustrated implementation.

FIG. 9 is a logical flow diagram generally showing one embodiment of a process for generating a dashboard for a group of users, according to one non-limited illustrated implementation.

FIG. 10 is a logical flow diagram generally showing one embodiment of a process for generating an activity for a group of personality profiles, according to one non-limited illustrated implementation.

FIG. 11 is a logical flow diagram generally showing one embodiment of a process for identifying a user personality profile based on a personality trait, according to one non-limited illustrated implementation.

FIG. 12 is a block diagram of a data fusion subsystem of the personality factor determination system, according to various embodiments described herein.

FIG. 13 is a block diagram of a data analytics subsystem of the personality factor determination system, according to various embodiments described herein.

FIG. 14 is a block diagram of an example processor-based device that may be used to implement at least a portion of the personality factor determination system or other system discussed herein (e.g., organization system, candidate system, content system).

### DETAILED DESCRIPTION

In the following description, certain specific details are set forth in order to provide a thorough understanding of various disclosed implementations. However, one skilled in the relevant art will recognize that implementations may be practiced without one or more of these specific details, or with other methods, components, materials, etc. In other instances, well-known structures associated with computer systems, server computers, and/or communications networks have not been shown or described in detail to avoid unnecessarily obscuring descriptions of the implementations.

Unless the context requires otherwise, throughout the specification and claims that follow, the word “comprising” is synonymous with “including,” and is inclusive or open-ended (i.e., does not exclude additional, unrecited elements or method acts).

Reference throughout this specification to “one implementation” or “an implementation” means that a particular feature, structure or characteristic described in connection with the implementation is included in at least one implementation. Thus, the appearances of the phrases “in one implementation” or “in an implementation” in various places throughout this specification are not necessarily all referring to the same implementation. Furthermore, the particular features, structures, or characteristics may be combined in any suitable manner in one or more implementations.

As used in this specification and the appended claims, the singular forms “a,” “an,” and “the” include plural referents unless the context clearly dictates otherwise. It should also be noted that the term “or” is generally employed in its sense including “and/or” unless the context clearly dictates otherwise.

The headings and Abstract of the Disclosure provided herein are for convenience only and do not interpret the scope or meaning of the implementations.

One or more implementations of the present disclosure are directed to systems and methods that provide improved functionality for recruitment, hiring and onboarding, and team management for organizations (e.g., employers). In at least some implementations, the systems and methods may utilize web-based technology that includes one or more of media capture, video sampling, peer collaboration, and text-based descriptors. Visual media may serve as proxy measures or representations of soft skills, such as personality traits, leadership, problem solving, teamwork, communication, interpersonal, flexibility/adaptability, work ethic, etc. The visual media may also be used to generate personality data which is used to generate one or more personality factor scores for the user.

The systems and methods may use personality theory to aid in generating the personality data and personality factor score for the user. Personality theory suggests that all individuals inhabit a unique set of traits that impact the ways in which they think, feel, behave, and interact with others with some degree of consistency. These theories, such as the Five Factor Model, Eysenck's Model of Personality, Cattel's 16PF Trait Theory, etc., assume that fundamental units of traits combine to form the core of individual's personalities and that a few basic higher order traits underlie more specific dispositions. For example, the Five Factor Model centers on five higher order traits, referred to as Extraversion, Neuroticism, Openness to Experience, Agreeableness, and Conscientiousness. The theory has become foundational to several industry standard personality assessments, because it is psychometrically viable and lends itself to administration on self-report questionnaires.

In at least some implementations, features or elements of a user profile, such as a social media profile, employee profile, etc., or of visual content user interface elements selected by a user, may be correlated with specific personality traits. Thus, the systems and methods described herein may use personality theory models, such as the Five Factor model, to predict which personality traits a user may have. In such embodiments, the elements of the user profile may be used to predict which personality traits the user may have by using personality theory models. In such embodiments, the systems and methods described herein may use AI/machine learning models in conjunction with, trained on, etc., personality theory models to determine the personality traits based on the user profile. Thus, in such embodiments, the systems and methods described herein are able to use a combination of user profiles, including imagery in the visual content user interface elements, artificial intelligence or machine learning models, and psychometrics to predict which personality traits a user may have, and determine a personality factor score for the user.

In some embodiments, the extent to which certain profile features are most predictive of particular traits is assessed by using validated personality assessments such as the NEO-PI 3. In some embodiments, these assessments are used to create a statistical model, artificial intelligence model, machine learning model, etc., which provides a prediction of the personality traits of a user.

A candidate, also referred to herein as a user, applicant, employee, employer, student, or job-seeker, may select one or more aspects/instances of visual media used to generate the personality data. In some implementations, each of the aspects/instances of visual media include or be provided with a text descriptor which is used to generate personality data for the candidate. The generated personality data is used to generate a personality factor score for the user. In at least some implementations, the personality factor score includes scores for one or more personality types, such as creativity, steadiness, persistence, cooperativeness, sociability, conscientiousness, openness, neuroticism, agreeableness, extraversion, etc. An indicator of a personality factor score may include one or more values such as one or more numerical scores, grades, etc.

Recruiters or hiring managers may use the personality scores generated for the user to evaluate candidates based on their personality score. In some implementations, the personality factor score is also used to search for users with particular personality factors. Additionally, the scores may be used to determine the user's effect on, compatibility with, etc., a group or team within the organization. For example, a recruiter may determine that a team within an organization requires a new member with a high level of creativeness. The recruiter may search for potential candidates based on their level of creativeness, and may see how each candidate's personality score would affect the team.

The systems and methods of the present disclosure advantageously allow organizations to determine, and search for individuals with, certain personality types. Additionally, the systems and methods of the present disclosure allow organizations to manage teams based on the personality types of those involved, and to create activities for team members to participate in to learn about the personality of other team members.

The systems and methods discussed herein may additionally be used to provide or enhance social networks, or may be used to identify future available positions for candidates based on their personality types. Although many embodiments are discussed in terms of job candidates, hiring, and organizational teams, embodiments are not so limited. The systems and methods discussed herein may also be used in the context of team dynamics, dating and relationships, education and living arrangements, and other situations where identifying and analyzing personality traits can provide useful information.

In at least some implementations, content may be selected or identified by the candidate or on behalf of the candidate (e.g. peer-sourcing). Similarly, an organization may submit content, identify (e.g. “like”) content, or directly or indirectly provide information indicative of the organization's desired content. In at least some implementations, the system may learn or identify the personality traits attractive to an organization by autonomously analyzing information associated with the organization, such as publically available information (e.g., website, social media, job descriptions), information provided by the organization, or personality traits of users associated with the organization.

The systems and methods provided herein may be used to determine a personality factor score for a user-personality profile. The personality factor score may be generated by presenting visual user interface elements to a user represented by the user-personality profile. In some embodiments, at least one of the visual user interface elements comprise one or more images. In some embodiments, at least one of the visual user interface elements comprise one or more videos. In some embodiments, at least one of the visual user interface elements comprise text. A user may select at least one of the visual user interface elements. The selected visual user interface elements are used to generate personality data for the user. In some embodiments, at least a portion of the data used to generate the personality data is obtained from one or more user profiles (e.g., social media profiles). In some embodiments, at least a portion of the data used to generate the personality data is obtained autonomously or from a secondary user. The personality data is used to generate a personality factor score which represents at least one personality trait of the user. In some embodiments, the personality factor score includes one or more scores. In some embodiments, the personality factor score is based on a personality model that accounts for one or more personality traits, such as creativity, steadiness, persistence, cooperativeness, sociability, conscientiousness, openness, neuroticism, agreeableness, extraversion, etc.

In some embodiments, personality data is obtained by presenting one or more words corresponding to one or more personality factors to the user. The personality data is altered based on the words selected by the user. In some embodiments, the words are presented in connection with one or more visual user interface elements. In some embodiments, the user provides textual data in free-form (e.g., words, phrases, sentences).

In some embodiments, the personality factor score is generated by applying the personality data to a machine learning model that has been trained to generate a personality factor score based on personality data.

In some embodiments, the systems and methods described herein include obtaining an indication of a personality trait and identifying one or more user-personality profiles within a plurality of user-personality profiles with the indicated personality trait. In some embodiments, the user-personality profiles are identified based on their personality factor score. In some embodiments, the plurality of user-personality profiles are profiles belonging to a user belonging to potential candidates for joining an organization.

In some embodiments, the systems and methods described herein are used to identify a user group based on the personality factor score of each of the user-personality profiles. In some embodiments, the group is identified by using data clustering. In some embodiments, the systems and methods described herein are sued to identify one or more roles within an organization. In some embodiments, the identified roles are used to identify at least one user-group, team, group, etc., of user-personality profiles. In some embodiments, a group personality profile is generated for the group.

In some embodiments, the systems and methods described herein are used to generate a dashboard for at least one identified user-group. The dashboard may include an indication of the personality factor score for each of the users belonging to the group. In some embodiments, the dashboard includes an indication of a group-personality profile for the group. In some embodiments, the systems and methods described herein are used to generate an activity for a user group based on the personality factor score of users within the group.

In some embodiments, the systems and methods described herein are used to identify correlations between user-personality profiles in the user group based on the personality data of each user-personality profile. In some embodiments, the correlations are identified by using a machine learning model trained to identify correlations based on personality data.

In some embodiments, the systems and methods described herein are used to determine how the group-personality profile changes based on the membership of a particular user. In some embodiments, the group-personality profile is altered based on changes to the personality profile of at least one user in the group. In some embodiments, the systems and methods described herein are used to identify trends in how the group-personality profile changes in time. In some embodiments, the trends include changes in the morale of the group.

These and other features are discussed further below with reference to the drawings.

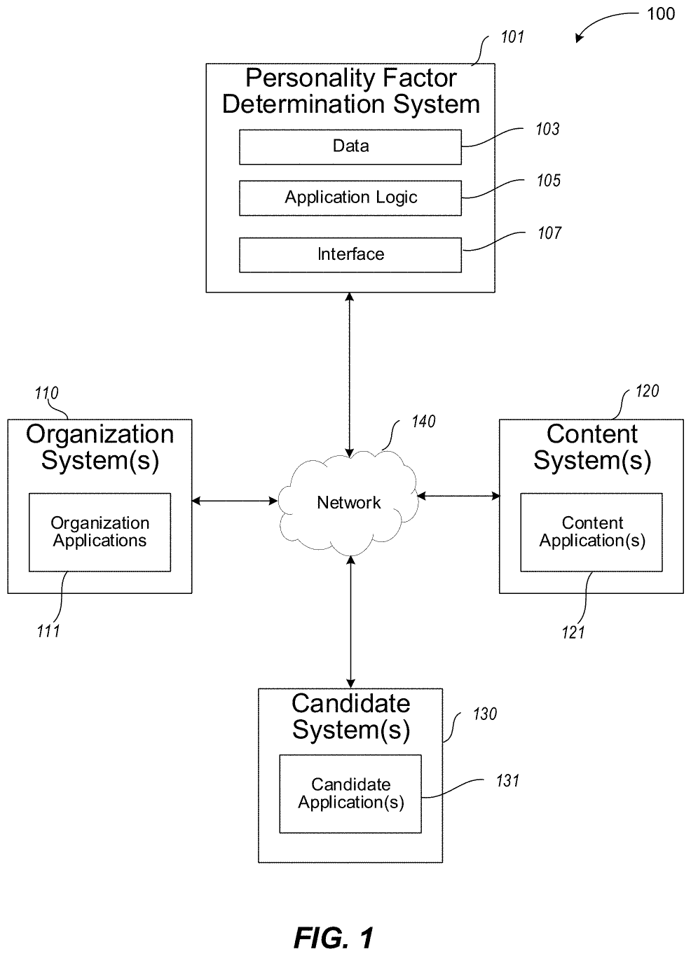

FIG. 1 is a diagram that illustrates an environment 100 in which a personality factor determination system 101 of the present disclosure may operate. The personality factor determination system 101 may interact with various other systems, such as organization systems 110, candidate systems 130, content source systems 120, etc., over a network 140 (e.g., the Internet).

As a non-limiting example, the personality factor determination system 101 may be generally organized in a three layer architecture that includes an interface layer 107, an application logic layer 105, and a data layer 103. Each component in FIG. 1 may represent a set of executable instructions and corresponding hardware, such as memory and processor(s), for executing the instructions, thereby forming hardware-implemented components operating as a special-purpose machine that performs a particular set of functions. For the sake of brevity, various functional components that are not necessarily important for providing an understanding of the subject matter of the present disclosure have been omitted. However, additional or fewer components may be used with the personality factor determination system to provide various functionality. Furthermore, the various components may be implemented in a single computing system or device (e.g., server computer, client device), or may be distributed across several computers in numerous configurations.

The interface layer 107 may include interface components, such as a web server, which receive requests from client computing devices and servers, such as organization system 110 executing organization applications 111, candidate system 130 executing candidate applications 131, or content system 120 executing content applications 121. In response to received requests, the interface 107 may communicate appropriate responses to requesting systems via the network 140. For example, the interface 107 may receive Hypertext Transfer Protocol (HTTP) requests, or other web-based, Application Programming Interface (API) requests.

The candidate system 130 may execute web browser applications or platform-specific applications (“apps”). For example, the candidate system 130 may interact with the personality factor determination system 101 via a web browser, an iOS® app, an Android® app, etc. Additionally, in at least some implementations, the candidate system 130 may perform some or all of the functionality of the personality factor determination system 101. Generally, the candidate system 130 may include a device that includes a display and a communication interface that allows the candidate system to communicate with the personality factor determination system 101 and other systems via the network 140. The candidate system 130 may include, for example, a personal computer, a smartphone, a wearable computer, a tablet computer, a personal digital assistant (PDA), a laptop computer, a desktop computer, a game console, a set-top box, etc.

The data layer 103 may include a database server that facilitates access to information storage units such as one or more databases. The databases may store various data, such as user profile data (including personality factor data), visual content data, textual data, event data, and other types of data.

The application logic layer 105 may include various application logic components which interact with the interface layer 107 and data layer 103 and implement some or all of the various functionality described herein. For example, the application logic 105 may include logic to facilitate the creation of device profiles, accessing content from content source systems 120 (e.g., systems operated by content providers), communicating with organization systems 110 (e.g., systems operated by hiring organizations, job posting organizations, etc.).

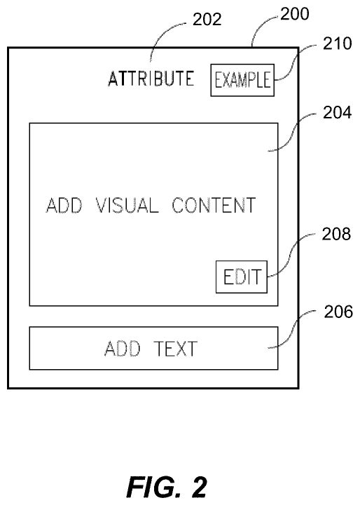

FIGS. 2-6 show various example interfaces of the personality factor determination system 101 of FIG. 1 that may be provided to allow the determination and analysis of personality factors for individuals and teams. The interfaces are provided as examples of the functionality provided by the personality factor determination system 101, and it should be appreciated that variations of the interfaces may be provided for different implementations or for different platforms (e.g., web browser versions versus mobile app versions). As discussed above, users may be prompted to select from one of a plurality of visual user interface elements. The user interface elements may include text, video, multiple images, etc. For example, a candidate may be presented with one or more of the user interface elements, of which the candidate selects at least one. Personality data is generated from the selected user interface elements. The personality data is used to generate a personality score for the user which describes at least one personality trait of the user.

As another example, an organization may search for candidates with certain personality traits as part of choosing a candidate to join the organization, such as in hiring, volunteering, etc. The personality scores may also be used to provide personality analysis for a group of users, candidates, members, etc. of an organization, such as a group-personality score, changes in the personality scores based on membership, etc.

FIG. 2 shows an example attribute selection interface 200 of the personality factor determination system 101 of FIG. 1, according to one illustrated implementation. The attribute selection interface 200 may be provided to a user responsive to the user indicating that they want to generate a personality score for at least one of their personality traits. In at least some implementations, an instance of the attribute selection interface 200 may be provided for each of a plurality of attributes that the personality factor determination system 101 captures or represents for the user when generating a personality data for a personality score. The number of attributes and selected visual user interface elements used to generate the personality data may be fixed (e.g., 2, 4, 8, or 20), or may be variable (e.g., determined by the user, determined by the organization, etc.). In some implementations, candidates may be able to represent new attributes or visual user interface elements that are not already included for selection in the personality factor determination system.

Non-limiting examples of attributes that may be captured in a digital profile by the personality factor determination system include sense of humor, attitude on life, activities, something inspiring, something creative, “show me something I may not know,” “show me something new,” “show me something unusual,” bucket list, your happy place, theme song, risk versus reward, “if you were a movie (or other something), what would you be?”, etc.

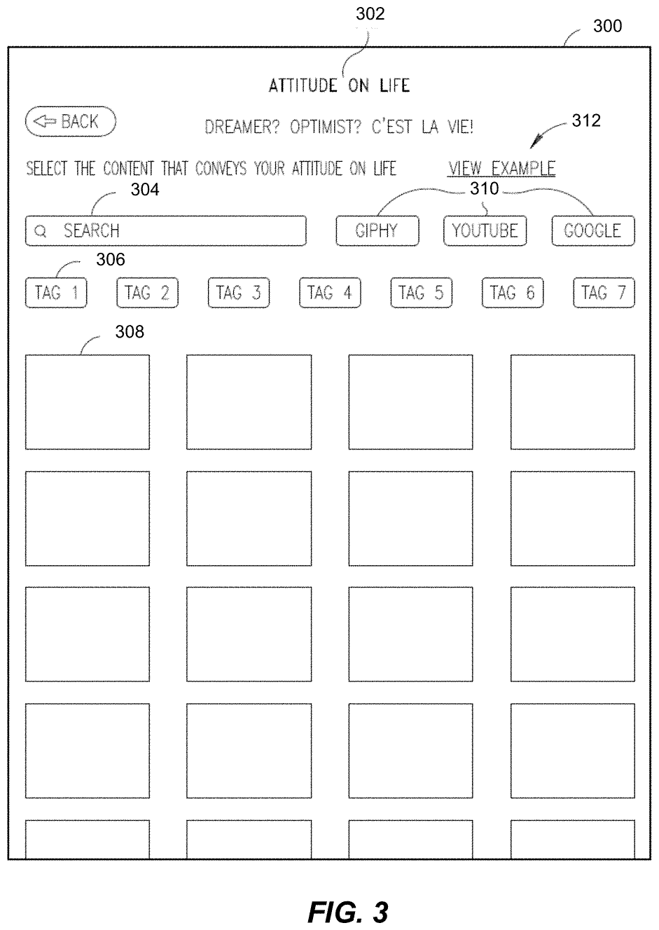

The attribute selection interface 200 displays the attribute name 202, and also includes a visual content window 204 which allows the candidate to add visual content for the attribute, and an add text window 206 for the candidate to add a textual descriptor that relates to the selected visual content or the attribute. To add visual content for the attribute, the candidate may select an edit button 208 positioned inside the visual content window 204, which causes a visual content selection interface 300 (FIG. 3) to be presented. To add a text descriptor, the candidate may select (e.g., click on, tap) the add text window 206. In some instances, the text descriptor may be limited to a determined length (e.g., 140 characters, 200 characters, 100 words). Further, in at least some implementations, the attribute selection interface 200 may alternatively or additionally include an interface that allows the user to select audio content, such as a song, a podcast, a poetry reading, etc. The attribute selection interface 200 may allow the user to create non-textual content to include in the attribute representation. Such content may include visual content (e.g., a drawing, an image, a video) or audio content (e.g., a statement, a song, a reading).

The attribute selection interface 200 may also include an example button 210 which, upon selection, presents an example attribute representation to the user that includes an example selected visual content and an example text descriptor. The provided example may be specific to the attribute currently being represented, or may be a common example for all attributes.

FIG. 3 is an example visual content selection interface 300, presented by the personality factor determination system 101 in some embodiments. As an example, the visual content selection interface 300 may be presented to the user responsive to selection of the edit button 208 on the attribute selection interface 200. The visual content selection interface 300 includes a title 302 that indicates the attribute to be represented. In this example illustration, the attribute is “attitude on life.” The visual content selection interface 300 also includes a search bar 304 that allows candidates to input text to be searched for in one or more visual content databases, and search results 308 may be presented to the user below the search bar. The visual content selection interface 300 may also include tags 306 that are generated based on the input text which allow the user to refine their search to include results that are associated with one or more selected tags. In some implementations, the visual content selection interface 300 may include content source selection buttons 310 that allow the candidate to specify one or more content sources (e.g., GIPHY, YouTube, Google) to be searched.

The user may select one of the results 308 to be used as visual content to generate personality data for the user. The user may select visual content and add a text descriptor for each of a plurality of attributes that are to be represented. The selected visual content, attribute, and any text descriptors for the visual content, will be used by the personality factor determination system as raw personality data for generating the personality score. Additionally, the selected visual content, attribute, and text descriptors may be used to generate personality data for the user.

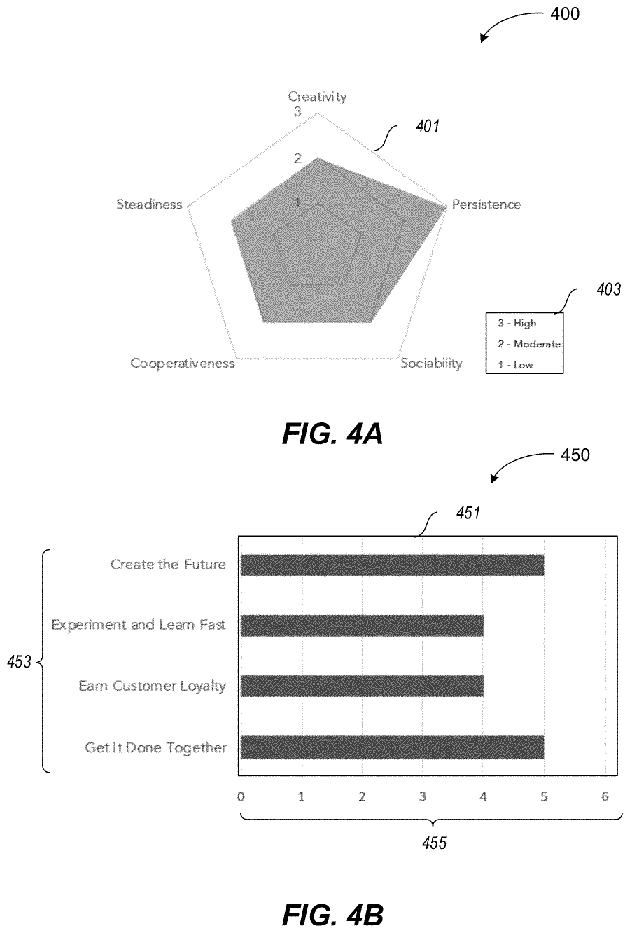

FIG. 4A is an example personality score screen 400 presented by the personality factor determination system 101 in some embodiments. The personality score screen 400 includes a personality graph 401 and a personality graph legend 403. The personality graph 401 includes a visual indication of personality scores for one or more personality traits for the user. In the example personality score screen 400, these traits are creativity, steadiness, cooperativeness, persistence, and sociability, however other personality traits may be indicated by the graph. The personality graph legend 403 assists the graph in indicating the impact of each trait on the overall personality of the user. In the example personality score screen 400, the personality graph legend 403 indicates that a score of 3 is high, 2 is moderate, and 1 is low. Thus, a user with a personality score of 3 in persistence has high persistence, and a user with a personality score of 1 in creativity has low creativity. In some embodiments, the personality graph legend 403 includes indicators other than numbers, such as letters, symbols, etc. In the personality graph 401: steadiness is a measure of a user's calmness and self-control; cooperativeness is a measure of a user's agreeability and motivation by the greater good; sociability is a measure of the user's interpersonal interest, ambition, and confidence; persistence is a measure of the user's dependability and conscientiousness; and creativity is measure of the user's desire to learn by experience and seek novelty.

FIG. 4B is an example personality trait prediction screen 450, presented by the personality factor determination system 101 in some embodiments. The second example personality trait prediction screen 450 includes a graph 451 which indicates predictions of a user's personality traits based on their personality score. The graph 451 has predicted traits 453 as its y-axis and scores 455 as its x-axis. The predicted traits 453 are traits, tasks, or soft-skills, which a user may have. The scores 455 are an indication of the user's proficiency with the predicted traits 453. In some embodiments, sores for the predicted traits are determined based on personality factor scores for base personality traits of the user. For example, in the graph 451: the “create the future” score is determined based on a combination of the user's creativity and persistence personality score's; the “experiment and learn fast” score is determined based on the user's creativity and cooperativeness personality scores; the “earn customer loyalty” score is determined based on the user's creativity and sociability personality scores; and the “get it done together” score is determined based on the user's cooperativeness and persistence personality scores. The combination of personality scores to obtain predicted traits can include simple mathematical operations, statistical analysis, the user of an artificial intelligence or machine learning model, etc. Like the personality graph legend 403, the scores 455 may include indicators other than numbers, such as letters, symbols, etc.

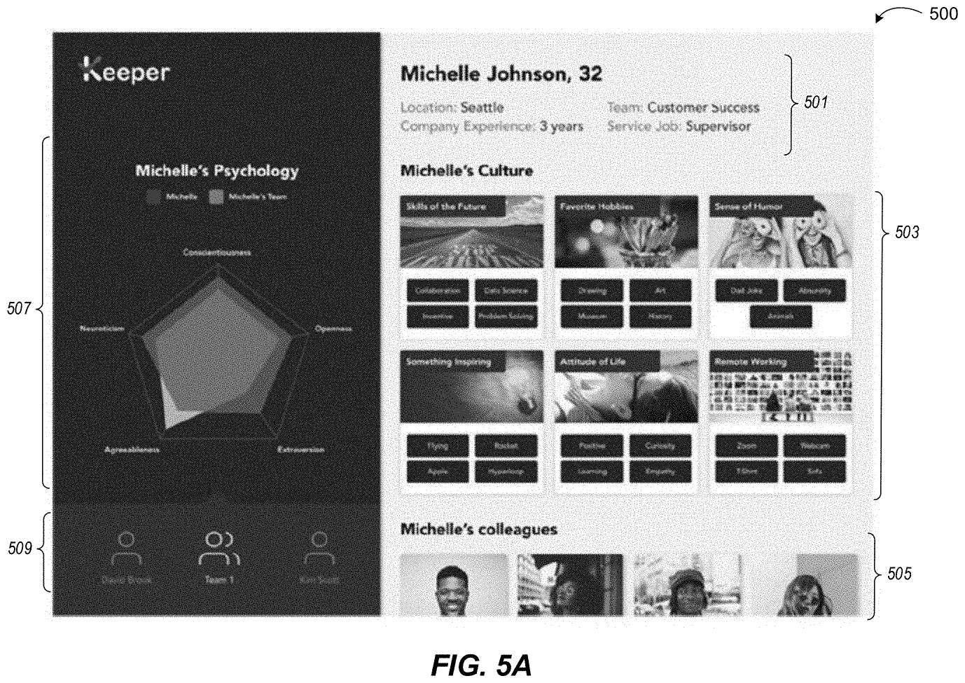

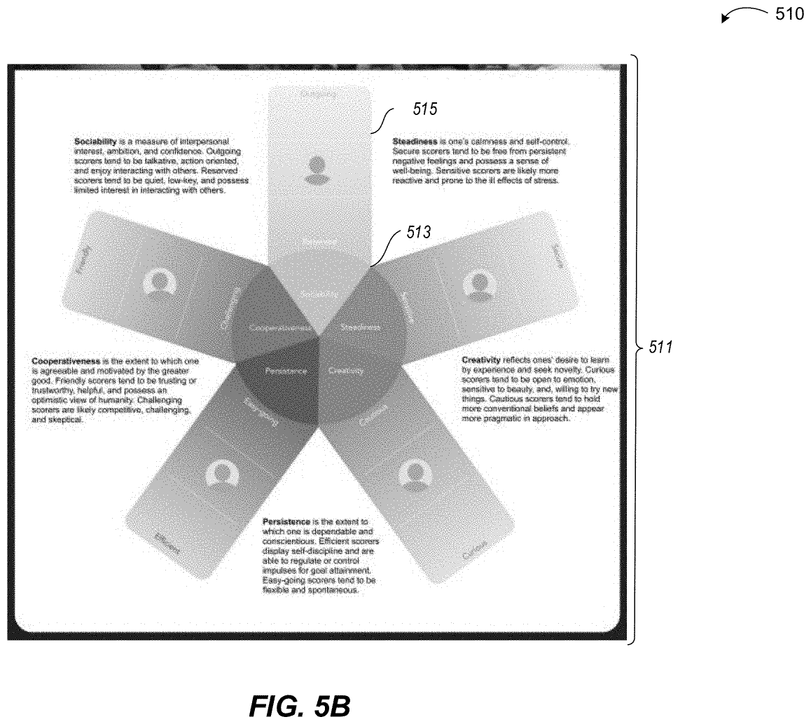

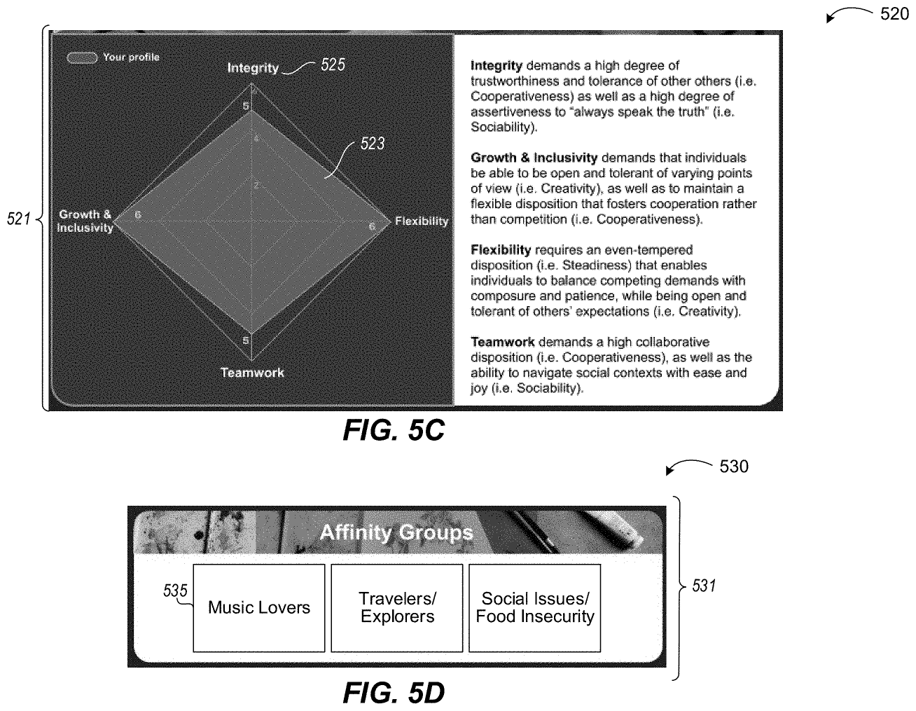

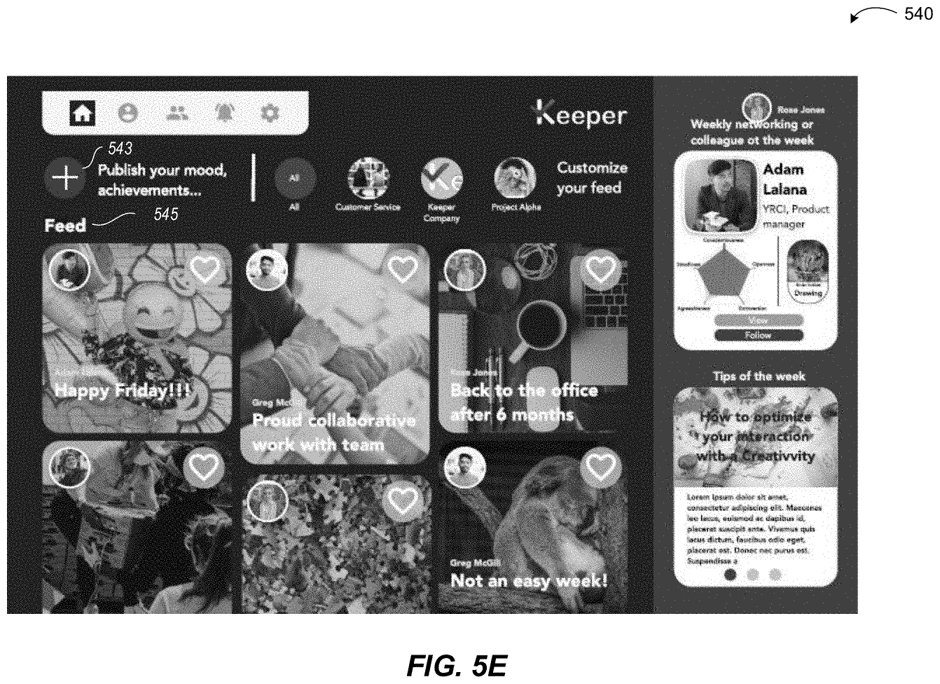

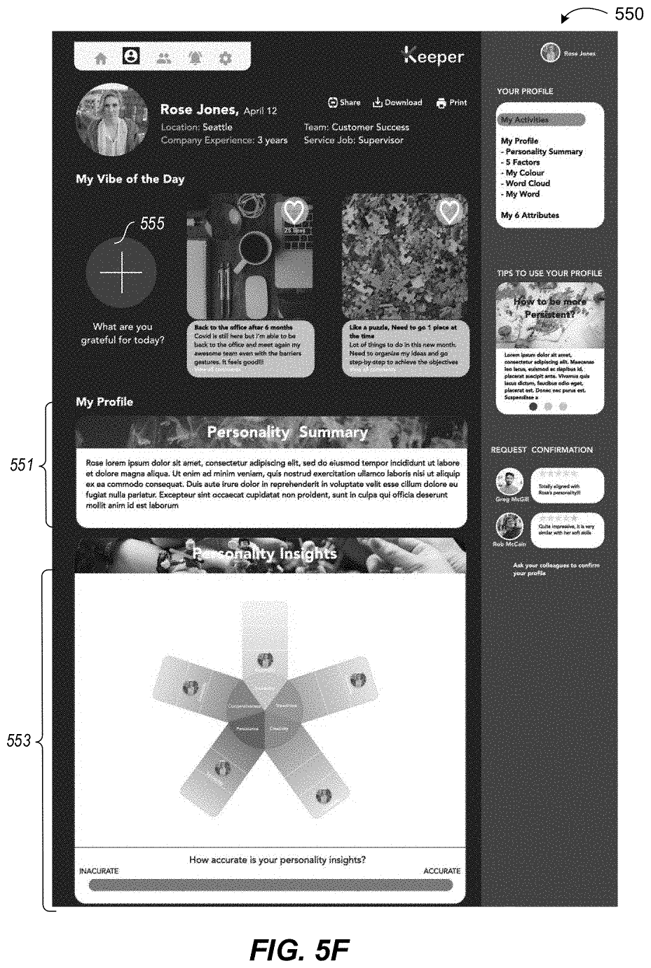

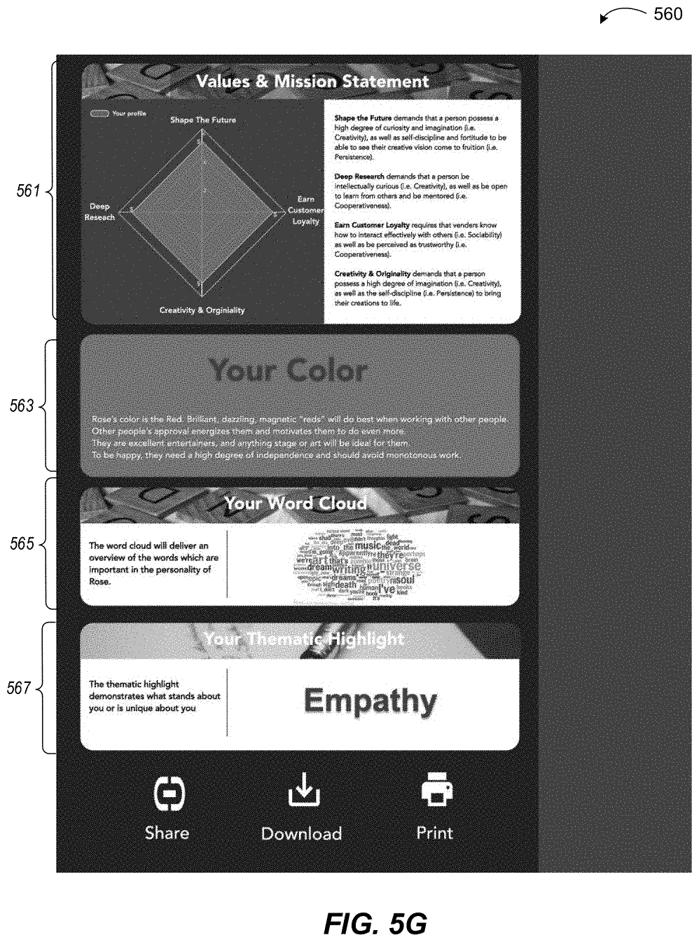

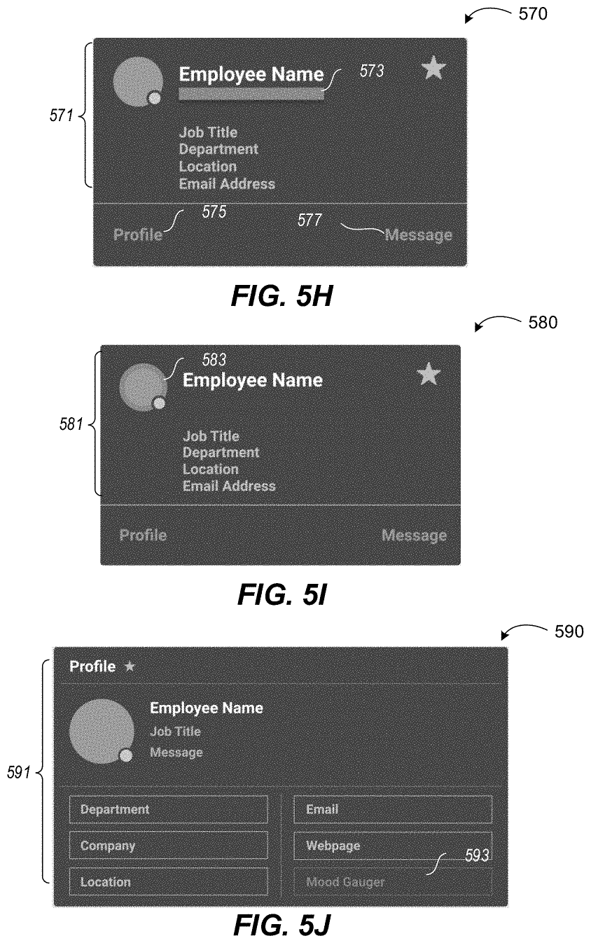

FIG. 5A is an example user dashboard 500 presented by the personality factor determination system 101 in some embodiments. The user dashboard 500 includes a variety of interfaces which indicate information regarding a user and their personality scores. The user information section 501 includes general information, such as demographic information, job information, etc., for a user. The culture section 503 includes user interface elements which indicate at least a portion of the personality data collected for the user. The colleagues section 505 indicates the user's colleagues within the organization. Additionally, the colleagues section 505 may be used to select a colleague, such as by interacting with a user interface element indicating the colleague, to view that colleague's personality scores, personality data, or a dashboard with the colleague's information. The psychology section 507 includes one or more graphs indicating the user's personality data or personality scores, such as the personality graph 401, the graph 451, or another graph indicating personality data or scores. The graphs may also be compared to graphs of a team, group, etc., that the user belongs to. The team section 509 includes an indication of one or more of the teams which the user belongs to. In some embodiments, interacting with an interface element that indicates one of the teams adjusts a graph in the psychology section 507 to overlay the team's personality score(s) on top of the user's personality score(s). In some embodiments, interacting with the interface element that indicates one of the teams may change the colleagues indicated in the colleague section 505. In some embodiments, interacting with the interface element that indicates one of the teams may cause a dashboard for the team to be shown.

FIG. 5B is an example personality factor graph screen 510, presented by the personality factor determination system 101 in some embodiments. The personality factor graph screen 510 is used by the personality factor determination system 101 to display a user's individual personality traits. The personality factor graph screen 510 includes a personality factor graph 511, which includes one or more factors 513 and one or more corresponding factor ranges 515. The one or more factors 513 may include factors such as: steadiness, a measure of calmness and self-control; sociability, a measure of interpersonal interest, ambition, and confidence; cooperativeness, a measure of the extent one is agreeable and motivated by the greater good; persistence, a measure of the extent one is dependable and conscientious; creativity, a measure of one's desire to learn by experience and seek novelty; or other personality factors or traits. The factor ranges 515 illustrates how much each personality factor make up a user's personality. Each personality factor included in the one or more factors 513 may have a corresponding factor ranges 515. For example, a sociability score may be measured on a scale of 1-5, where a score of 1 indicates the user is reserved, and a score of 5 indicates the user is outgoing. The factor ranges 515 indicates that the user has a mid-range score, such as 3 on a scale of 1-5. This score indicates that the user is somewhere in the middle of completely outgoing and completely reserved.

FIG. 5C is an example values graph screen 520, presented by the personality factor determination system 101 in some embodiments. The values graph screen 520 may include a values graph 521. The values graph 521 includes an indication of a user's values calculated by the personality determination system 101 based on the personality factor scores for two or more personality traits of a user. The values graph may indicate one or more value scores 523 for one or more values 525. The one or more values 525 may include: integrity, a measure of the user's trustworthiness and tolerance of others; growth and inclusivity, a measure of the user's ability to be open and tolerant; flexibility, a measure of the user's disposition, composure, and patience; teamwork, a measure of the user's collaborativeness and ability to navigate social context; and other values which can be determined based on two or more personality factor scores. The one or more value scores 523 are calculated based on the personality factor scores for two or more personality traits, for example, a user's integrity may be calculated based on the user's cooperativeness and sociability personality factor scores.

FIG. 5D is an example affinity group screen 530, presented by the personality factor determination system 101 in some embodiments. The affinity group screen 530 displays a user's affinity groups, which are groups related to the user's interests and personality traits. The affinity group screen 530 includes an affinity group list 531 and one or more affinity group boxes 535. The affinity group list 531 indicates each affinity group the user belongs to by including one or more affinity group boxes 535. The affinity group boxes 535 each indicate one or more affinity groups in which the user belongs. The affinity groups may be chosen by the user. In some embodiments, the personality determination system 101 predicts, or suggests, an affinity group for the user based on the user's personality factor scores. For example, the personality factor determination system 101 may predict the user's affinity group by using a machine learning model to predict affinity groups based on user personality factor scores. The personality factor determination system 101 may also predict the user's affinity group by comparing the user's personality factor scores to other users included in the affinity group. For example, the personality factor determination system 101 may suggest to the user that they join the “music lovers” affinity group based on a determination that the user's personality factor scores are similar to users already in the “music lovers” affinity group.

FIG. 5E is an example “mood gauger” and feed screen 540, presented by the personality factor determination system 101 in some embodiments. The mood gauger and feed screen 540 includes a mood publish button 543 a feed 545, and a person of the week display 547. When a user activates the mood publish button 543, the personality factor determination system 101 prompts the user to indicate a mood, achievement, or other shareable information, to other users of the personality factor determination system 101. In some embodiments, the personality factor determination system 101 predicts a user's mood based on information indicated by the user through the mood publish button 543. For example, the user may publish that they “received a major award” within the company. The personality factor determination system 101 may use this information, along with a personality factor score, to predict the user's mood or how they are feeling, such as “content”, “joyful”, “accomplished”, or other moods or feelings. The personality factor determination system 101 may use a machine learning model trained to predict a user's mood based on their personality factor scores and input from the user. The personality factor determination system may also predict the user's mood based on the mood of other users who shared similar information and who have similar personality factor scores to the user.

The feed 545 indicates information describing the mood of other users within the organization, or who are connected with the user in some way, for example, co-workers, team members, friends, professional or non-professional connections, etc. In some embodiments, when a user interacts with the information describing the mood of other users, the personality factor determination system 101 displays a profile for the user, such as the user dashboard 500, an insight report screen, such as the example insight report screen 550, etc.

FIG. 5F is a first portion of an example insight report screen 550, presented by the personality factor determination system 101 in some embodiments. The first portion of an insight report screen 550 includes data generated by the personality factor determination system 101 based on the personality factor scores. A second portion of the insight report screen is discussed with regards to FIG. 5G. A user may view the insight report of another user, or of themselves, to learn about the other user or themselves. The first portion of an insight report screen 550 includes a personality summary 551, a personality factor graph 553, and a mood publish button 555. The personality factor graph 553 is similar to the personality factor graph 511. The mood publish button 555 is similar to the mood publish button 543. The personality summary 551 includes information indicating a summary of the user's personality. The summary may be generated by the personality factor determination system 101 based on the user's personality factor scores. The user may input information to be included in the personality summary.

The insight report may also include other information related to, or shared by, the user, such as favorites, e.g. foods, sports, hobbies, etc., demographic information, profile tips, connection requests, current moods, and other information related to or shared by the user. The insight report may further include a rating for the report, which can be submitted to by the user, the user's colleagues, other users accessing the report, etc. The rating may represent the accuracy of the insight report in describing the user's personality.

FIG. 5G is a second portion of an example insight report screen 560, presented by the personality factor determination system 101 in some embodiments. The second portion of the example insight report screen 560 includes a second portion of the insight report which may be generated by the personality factor determination system 101. The second portion of the insight report screen 560 may include a values graph 561, a personal color 563, a word cloud 565, and a thematic highlight 567. The values graph 561 is similar to the values graph 521. The color 563 indicates a color representing the user's personality. The personality factor determination system 101 may generate the color representing the user's personality based on the personality factor scores of the user. For example, the personality factor determination system 101 may use a machine learning model trained to generate a color for a user based on at least their personality factor scores. The personality factor determination system 101 may determine a user's color based on colors for other users with similar personality factor scores.

The word cloud 565 includes words which describe, are important to, etc., the personality of the user. The words in the word cloud may be generated by the personality factor determination system. For example, the personality factor determination system 101 may use a machine learning model trained to choose words describing a user's personality based on at least their personality factor scores. The personality factor determination system 101 may choose words describing a user's personality based on words describing other users with similar personality factor scores.

The thematic highlight 567 includes a word or phrase which describes the user. The thematic highlight may be generated by the personality factor determination system. For example, the personality factor determination system 101 may use a machine learning model trained to determine a thematic highlight for a user based on at least their personality factor scores. The personality factor determination system 101 may determine a thematic highlight for a user based on thematic highlights chosen for other users with similar personality factor scores.

FIG. 5H is a first example personality integration screen 570, presented by the personality factor determination system 101 in some embodiments. The first example personality integration screen 570 depicts an integration of some of the features of the personality determination system 101 in other applications and software. The first example personality integration screen 570 includes a profile information section 571, a mood gauger display 573, a profile button 575, and a message button 577. The profile information section 571 includes information describing a user's demographic information, job information, contact information, etc. The mood gauger display 573 indicates a mood for the user generated by the personality determination system, such as a mood generated as a result of a user publishing information using the mood publish button 555 or mood publish button 543. The mood gauger display 573 may include a color representation, text representation, or other representation, of the user's mood.

When a user interacts with the profile button 575 the personality determination system 101 displays a profile for the user indicated by the profile information section 571, such as an insight report, a user dashboard, or other profile for the user. When a user interacts with the message button 577 the personality determination system 101 displays a messaging window used to message the user indicated by the personality information section 571. The message window may include information regarding at least one of the user's personalities, such as a portion of the insight report, a personality factor graph, such as the graphs discussed with regards to FIGS. 5A, 5B, and 5C, affinity groups, favorites, or other information related to the user's personality information. For example, if a first user indicates that they enjoy Thai food, the personality factor determination system 101 may display that the first user enjoys Thai food to a second user, who can then suggest Thai food for lunch.

FIG. 5I is a second example personality integration screen 580, presented by the personality factor determination system 101 in some embodiments. The second example personality integration screen 580 includes a profile information section 581, similar to the profile information section 571, and a mood gauger 583. The mood gauger 583 is similar to the mood gauger 573, except the mood gauger 583 is displayed around a user's profile picture rather than underneath the user's name.

FIG. 5J is a third example personality integration screen 590, presented by the personality factor determination system 101 in some embodiments. The third example personality integration screen 590 includes a profile information section 591, similar to the profile information section 571, as well as a mood gauger 593 similar to the mood gauger 573.

Each of the integration screens 570, 580, and 590, are example integration screens depicting how a portion of the personality determination system 101 may be integrated into another applications, such as RingCentral or any other Human Capital Management systems, social media applications and websites, etc. The personality determination system 101 may be directly integrated into the other application, or may be accessible to the other application, such as being another application running the same computing system, device, server, etc., by being accessible through a web interface, or other methods of accessing information between one or more computing devices. The other applications may also include methods to search for, or access, insights, insight reports, and profiles for users of the personality factor determination system 101, such as integration with a chatbot, a calendar, etc. In embodiments where a chatbot is used, the chatbot may send user profiles to a user based on identifying user profiles for participants in a meeting, members in organizations, etc.

For example, in one use case, a client and a vendor may create user profiles by using the personality factor determination system 101. The client and vendor may then share their profiles with each other and use the profiles, insight reports, and other data generated by the personality factor determination system 101 to better communicate with each other, such as, as an icebreakers, to determine which people in each organization may work well with each other, to better understand each other, etc.

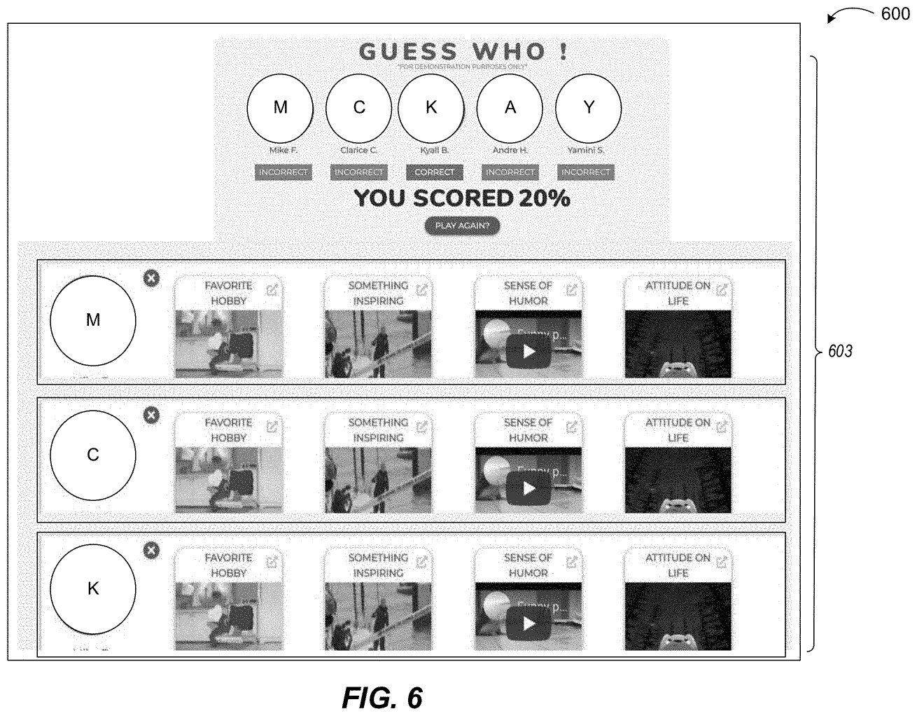

FIG. 6 is an example activity screen 600, presented by the personality factor determination system 101 in some embodiments. The example activity screen 600 includes an activity section 603. The activity section 603 includes user interface elements for participating in an activity generated by the personality factor determination system based on the personality score for each of the users participating in the activity. In some embodiments, the users participating in an activity are part of a user group. The activity generated by the personality factor determination system may include: a matching game for guessing group member's personality factors, a game designed to assist users in the group in developing soft skills, a game designed to assist users in the group in working together based on their personality differences, a game designed to allow users in the group to learn about each other, etc. In the example activity screen 600, the activity section 603 includes a matching game where users guess which personality attributes belong to other users in a group, however, other games designed to inform users about each other may be used by the personality factor determination system 101.

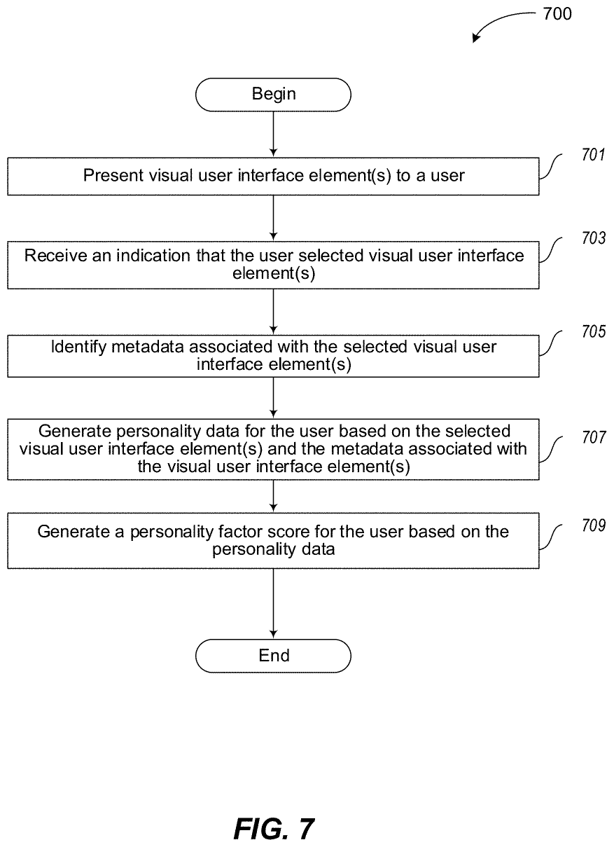

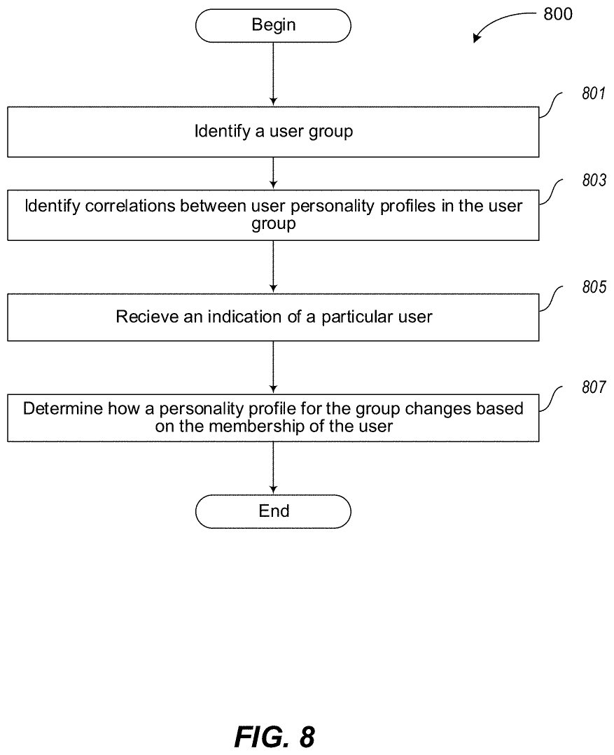

The operation of certain aspects of the disclosure will now be described with respect to FIGS. 7-11. In at least one of various embodiments, processes 700-1100 described in conjunction with FIGS. 7-11, respectively, may be implemented by or executed on one or more computing devices, such as processor-based device 1400 in FIG. 14 or other computing systems.

FIG. 7 is a logical flow diagram generally showing one embodiment of a process 700 for generating a personality factor score for a user, according to one non-limited illustrated implementation.

Process 700 begins, after a start block, at block 701, where one or more visual user interface elements are presented to a user. As discussed herein, an attribute selection interface 200 in FIG. 2 may be displayed to the user, which requests the user to provide content for various personal attributes. Examples of such attributes include, but are not limited to, sense of humor, attitude on life, activities, something inspiring, something creative, “show me something I may not know,” “show me something new,” “show me something unusual,” bucket list, your happy place, theme song, risk versus reward, “if you were a movie (or other something), what would you be?”, etc.

Process 700 proceeds to block 703, where an indication that the user selected one or more visual interface elements is received. Process 700 continues at block 705, where metadata associated with the selected visual user interface element(s) are obtained. In various embodiments, the visual interface elements include metadata which indicate their weight in determining a personality factor score for the user. In some embodiments, at least a portion of the metadata is obtained from a database, data structure, data lake, etc., which includes metadata for one or more of the visual user interface element(s). In some embodiments, the metadata includes text data indicating the weight of the visual content for generating a personality score.

Process 700 continues to block 707 where the personality factor determination system generates personality data based on the selected visual user interface elements and the metadata associated with the user interface elements. In some embodiments, the metadata is input into a machine learning model trained to generate personality data based on the metadata.

Process 700 continues to block 709 where the personality factor determination system generates a personality factor score for one or more personality traits of the user based on the personality data. In some embodiments, the personality factor score is generated by converting the generated personality data into a set of scores. In some embodiments, the set of scores is further manipulated, such as by using a statistical model or statistical analysis, to obtain a personality factor score for one or more personality traits of the user. In some embodiments, the personality data is input into an artificial intelligence or machine learning model trained to generate a personality factor score for one or more personality traits of a user based on personality data. In some embodiments, the personality factor score is based on a personality theory model. In some embodiments, the personality factor scores for a plurality of particular traits are combined to create a prediction of another, more complex, personality trait.

After block 709, process 700 ends.

FIG. 8 is a logical flow diagram generally showing one embodiment of a process 800 for identifying a user group and determining how a group personality profile changes based on membership of a particular user, according to one non-limited illustrated implementation.

Process 800 begins, after a start block, at block 801, where the personality factor determination system identifies a group of users. In some embodiments, the group of users is a group, team, cohort, etc., belonging to an organization. In some embodiments, the group is identified by the personality factor score for one or more traits of one or more users. For example, users with similar personality factor scores for a certain trait, different personality factor scores for a certain trait, etc., may be selected to be a part of the user group.

Process 800 proceeds to block 803, where the personality factor determination system identifies correlations between user personality profiles in the user group. In some embodiments, the correlations are determined based on the personality factor scores of each user in the user group. In some embodiments, the correlations are determined based on the personality data of each user in the group. In some embodiments, the correlations are used to determine a personality profile for the group.

Process 800 continues at block 805, where the personality factor determination system receives an indication of a particular user. The process continues to block 807, where the personality factor determination system determines how a personality profile for the group changes based on the membership of the particular use, including whether the user is removed, added, etc.

After block 807, process 800 ends.

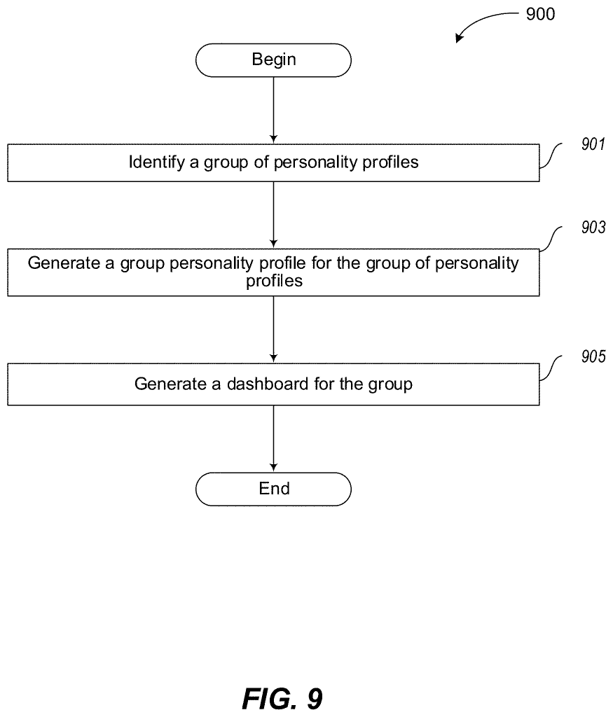

FIG. 9 is a logical flow diagram generally showing one embodiment of a process 900 for generating a dashboard for a group of users, according to one non-limited illustrated implementation.

Process 900 begins, after a start block, at block 901, where the personality factor determination system identifies a group of user personality profiles. In some embodiments, the group of user personality profiles belong to one or more users which belong to a team, group, cohort, etc., of an organization. In some embodiments, the group of user personality profiles are chosen based on one or more personality factor scores of the user personality profiles, such as based on similarities in personality factor scores, differences in personality factor scores, correlations between personality factor scores, etc.

Process 900 proceeds to block 903, where the personality factor determination system generates a group personality profile for the identified group of user personality profiles. In some embodiments, the group personality profile is generated based on the personality factor scores of each of the user personality profiles in the group. In some embodiments, the group personality profile is generated based on correlations identified between user personality profiles in the group.

Process 900 proceeds to block 905, where the personality factor determination system generates a dashboard for the group. In some embodiments, the dashboard includes one or more of: an indication of the users represented by the user personality profiles in the group, an indication of the group's personality profile, an indication of the group's personality factor score, an indication of the personality profiles for each user in the group, an indication of the personality factor scores for each user in the group.

After block 905, process 900 ends.

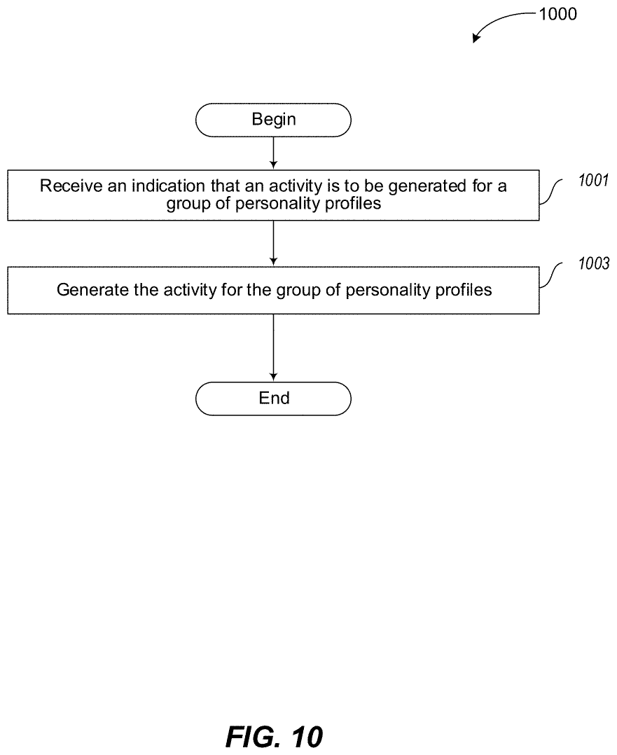

FIG. 10 is a logical flow diagram generally showing one embodiment of a process 1000 for generating an activity for a group of personality profiles, according to one non-limited illustrated implementation.

Process 1000 begins, after a start block, at block 1001, where the personality factor determination system receives an indication that an activity is to be generated for a group of personality profiles. In some embodiments, a dashboard generated for a group includes user interface elements which send an indication that an activity is to be generated for the group. In some embodiments, the activity to be generated includes a matching activity which prompts users to guess which personality traits belong to other users in the group.

Process 1000 proceeds to block 1003, where the personality factor determination system generates the activity for the group of user personality profiles. In some embodiments, the personality factor determination system manages the activity.

After block 1003, the process ends.

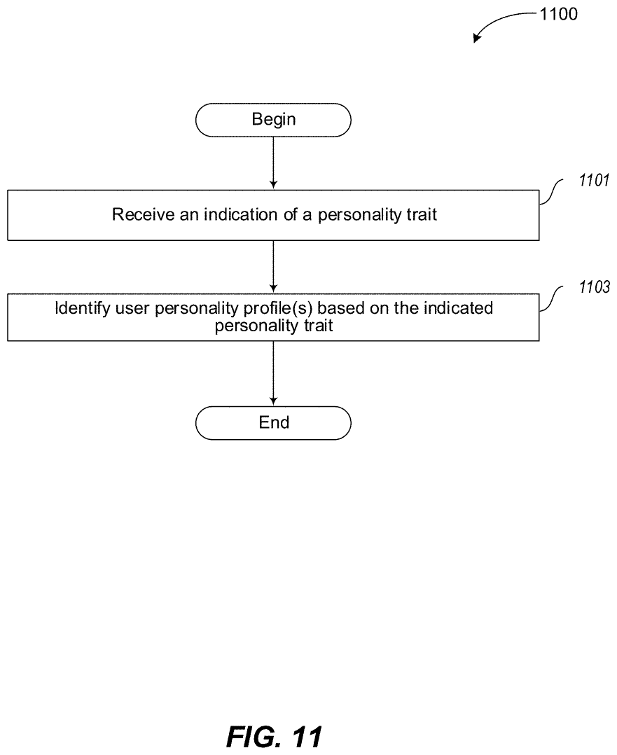

FIG. 11 is a logical flow diagram generally showing one embodiment of a process 1100 for identifying a user personality profile based on a personality trait, according to one non-limited illustrated implementation.

Process 1100 begins, after a start block, at block 1101, where the personality factor determination system receives an indication of a personality trait. In some embodiments, the indication of a personality trait is obtained through the use of a search bar, search function, as part of search criteria, etc.

Process 1100 proceeds to block 1103, where the personality factor determination system identifies one or more user personality profiles based on the indicated personality trait. In some embodiments, the indicated personality trait includes a personality factor score, and the user personality profiles are identified based on at least the personality factor score and the indicated personality trait.

After block 1103, process 1100 ends.

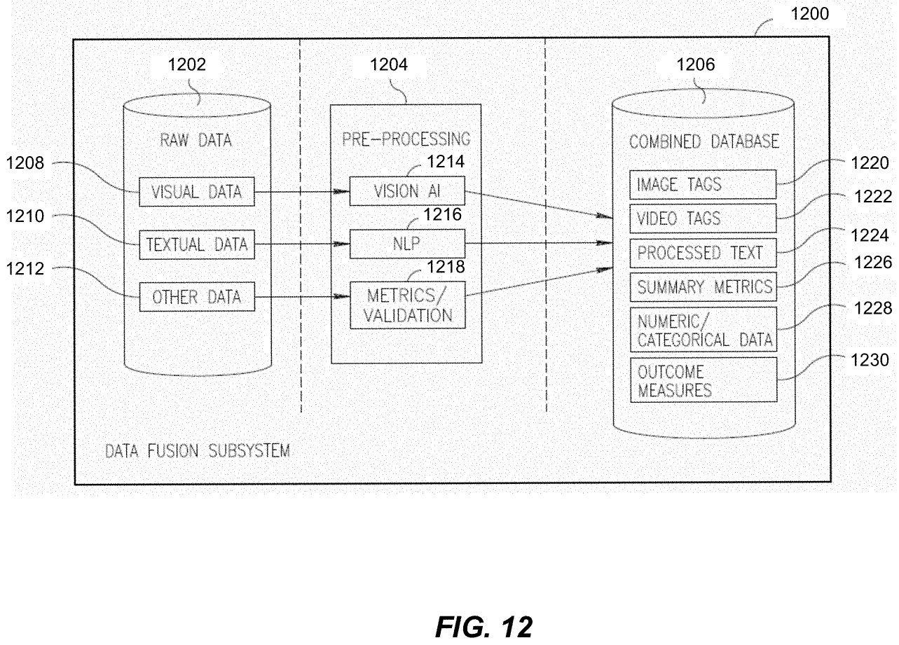

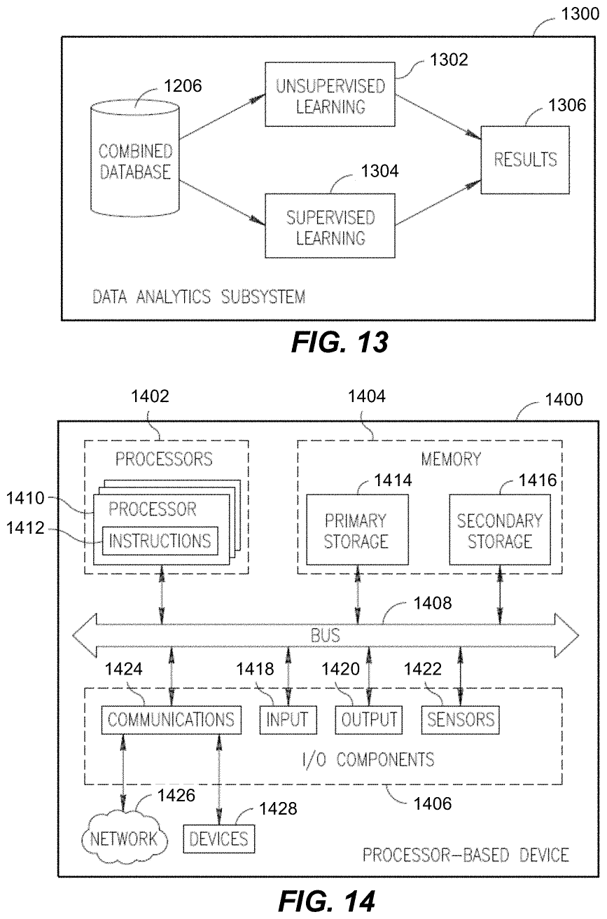

FIG. 12 is a block diagram of a data fusion subsystem 1200 of the personality factor determination system 101, according to various embodiments described herein. FIG. 13 is a block diagram of a data analytics subsystem 1300 of the personality factor determination system 101, according to various embodiments described herein. The data fusion subsystem 1200 and the data analytics subsystem 1300 may be used to provide important insight to users and user groups.

For example, in at least some implementations multiple data analysis concepts are combined, such as data fusion (e.g., drawing from several disparate data sources), the use of Natural Language Processing (NLP) methods to identify key concepts in a document database, an interface which collects feedback from users, and predictive models based on all of the above to generate personality data and personality factor scores. These and other features are discussed further below.

The data fusion subsystem 1200 may include a raw data repository or layer 1202 (“data lake”), a cleaning or pre-processing layer 1204, and a resulting combined database 1206. The raw data repository or layer 1202 may include visual data 1208, textual data 1210, and other data 1212. Visual data 1208 may include images or video. Textual data 1210 may include digital profile data, data entered by a user, data entered by someone with knowledge of the users, resume items, peer recommendations, etc. Other data 1212 may include usage data, history data, social network inputs, outcome measures, etc.

Many of the data sources, such as the use of visual imagery, media links, and natural language comments and descriptions, may require pre-processing to be suitable for use in an analytical framework, especially in an automated way. As an example, the pre-processing layer 1204 may include a vision artificial intelligence (AI) module 1214 that automatically tags the visual input data 1208. The pre-processing layer 1204 may further include an NLP module 1216 that processes the textual input data 1210. The pre-processing layer 1204 may further include a metrics/validation module 1218 that computes proprietary summary metrics, and provides validation or other checks for numeric and categorical data.

The resulting combined database 1206 may store various processed data, such as image tags 1220, video tags 1222, processed text 1224 (e.g., stemmed, tokenized, etc., with order preserved), summary metrics 1226, numeric/categorical data 1228, outcome measures 1230, etc. The combined database 1206 may be accessed by the data analytics subsystem 1300 to allow the data analytics subsystem to perform various analytics, as discussed further below.

Data sources may include information directly supplied by users and organizations, system usage data, feedback over time from users and organizations, external data sources disclosed by users, etc. External data sources may require specific approval from users before data is accessed, and some sources may be more sensitive than others. Several non-limiting example external data sources that may be used by the personality factor determination system are discussed below.

One external data source that may be used is LinkedIn or another business networking site. For example, a user may choose to supply a link to their LinkedIn profile to the personality factor determination system. The public information on a LinkedIn profile may contain endorsements (e.g., keywords), references (e.g., written narratives), and other categories which may not be present on a submitted resume. The personality factor determination system may process this information into a rich data set which is used for analysis or other action. For example, the personality factor determination system may use NLP methods to scan the references to identify key concepts about the user. This may include keyword searches or a machine learning process which automatically identifies key concepts associated with certain personality traits. Note that a concept might be more complicated than just the presence of a word or phrase, the concept may involve the order or absence of certain phrases, interaction with other attributes of the job-seeker, or attributes of the person writing the reference (e.g., whether they were a colleague or a supervisor).

Another data source may include background checks. This is a regulated area which generally requires specific permission from a user and clear disclosures about how the information will be used. Use of background check information can be very sensitive, and therefore in at least some instances may be avoided in predictive models.

Another data source may include social networks. As discussed briefly above, the personality factor determination system may provide tools which allow a user to share their personality profile with other users, have friends comment or participate in constructing their personality profile, etc. Apart from the data collected directly in this process, the personality factor determination system may also examine public information of the users who participate, subject to appropriate permissions.

Other data sources may include personal websites, portfolios, blogs, etc. When a user shares these resources with the personality factor determination system, they provide extensive information in a very unstructured format. The personality factor determination system may use NLP to scan this unstructured data and convert the data into usable metrics and key terms. The personality factor determination system may be designed to focus specifically on job-relevant content to provide the most relevant results.

As shown in FIG. 13, the data analytics subsystem 1300 may input data from the combined database 1206 into one or more unsupervised learning modules 1302 or one or more supervised learning modules 1304 to generate results data 1306.

Non-limiting examples of unsupervised learning methods include clustering and classification, NLP, anomaly detection, principal component analysis (PCA), singular value decomposition (SVD), time series, imputation, hypothesis testing, etc. Clustering and classification methods may include hierarchical agglomerative, nearest neighbor, k-means (parametric), neural networks, support vector machine (SVM), regression-based methods (e.g., logistic regression, random forests), tree-based methods (e.g., classification and regression Trees (CART)), etc.

Non-limiting examples of supervised learning methods include prediction and classification, survival analysis, power analysis, etc. Example prediction methods include regression-based methods, neural networks, hidden Markov models, etc. In at least some instances, there may be overlap between supervised and unsupervised learning methods because it may be advantageous to predict attributes other than an overall outcome or single ranking summary for each candidate.

The results data 1306 may include, for example, personality traits, scores for each personality trait, enhanced understanding of a user group, scores for personality traits present in a user group, identification of trends over time for a user or user group, identification of key drivers of personality traits, visualization or interpretation of structure in the data, etc.

The data analytics subsystem 1300 may provide insight to a user's personality. Over time, a profile page showing a user's personality profile, as well as interests and social connections, activities, and group membership and personality profiles, can convey information regarding a user's personality with regards to an organization. This information may be collected and archived for later analysis. Even with limited data, unsupervised clustering methods may be used to develop an understanding of the set of users and how they relate to roles within organizations, a personality profile for an organization, group and team dynamics, etc.

The personality factor determination system may utilize personality traits and scores to examine how a user relates to a team or organization's overall personality profile. The examination of how a user relates to a team or organization's overall personality profile may include the addition or removal of the user to the team or organization, correlations between the user's personality profile and the team or organization's personality profile, etc. Although in at least some implementations multiple measures may be combined into a single rank number (e.g., a “personality factor score”), in at least some implementations, a profile summary may be presented which conveys a significant amount of information about a user, including a personality factor score for multiple traits, traits created by combining other traits, etc.

The user profile page may present statistics, personality information, and other information about a user over the course of their history using the personality factor determination system. The profile summary page may be divided into multiple areas of information, such as a user personality profile section, user demographics section, and user team information section.

The user personality profile section may include some or all personality information for the user. This may include personality scores, personality factors, personality attributes of the user, other personality data, etc.

The user team information section may include the information regarding team information for one or more teams which the user is a part of. The team information may include other users which belong to the team, a team personality profile, team personality data, team personality traits, etc.

The personality factor determination system may capture various user data, in addition to data describing visual user interface elements selected by the user, for analysis including, for example, standard date/time stamps, geo-location stamps, login data, profile edits, etc. The personality factor determination system may also capture various organization data for analysis including, for example, organization driven events (e.g., profile shares, organization team information, organizational roles, user personality profiles), organization driven behavior (e.g., change in membership for a team, data obtained from team activities, changes in personality profiles of teams or users over time), etc.

Unsupervised clustering methods allow insight into natural groupings in the data. The personality factor determination system may use such methods to explore how particular profile elements are related to different personality traits. The system may also use anomaly detection (or fraud detection) methods to identify users or user personality profiles which stand out in some way.

The personality factor determination system may use ranking methods to match up users in a team in a game setting. In a customer service setting, ranking methods can combine personality scores in a way that is robust to outliers (e.g., tempering the impact of an occasional bad day) or which draws extra scrutiny to anomalies. Predictive models may be used to compare a user to previous users with known outcomes, which allows generation of rankings for who is likely to have a good outcome. For example, a personality profile for a potential new member to a team may be compared to a personality profile of a former team member to determine whether the new member is a good fit for the team. This may be tailored to a particular job category, employer, organization, position, etc.

The personality factor determination system may use recommendations and peer reviews as part of a credibility assessment. The content of these narratives may be analyzed using NLP, which allows for decomposition of the narrative into a set of elements which can form a foundation for clustering methods and predictive models.

The data analytics subsystem 1300 may also provide insight into a user's soft skills. As discussed above, it is difficult to quantitatively assess soft skills such as reliability, professionalism, focus, courtesy, teamwork, etc. These skills may be used to enhance, confirm, adjust, etc., personality factor scores and personality traits for a user or team. The personality factor determination system may use data science to solve this problem by surfacing consistent mentions or signifiers of soft skills in unstructured data, such as recommendations written for a user, narratives written by the user, or external data supplied by the user such as blogs or profiles on other sites. When an appropriate data set is available, the system may also look for quantitative predictors which are associated with good soft skills outcomes.

The system may initially generate a list of terms related to soft skills and personality traits, which provides a target space to use with NLP tools. As an example, the system may use regular expressions to scan through available data and then apply unsupervised clustering methods. Then, the system may use Term-Frequency and Inverse Document Frequency (TF, IDF, and TF/IDF) analysis to examine the term list in the context of unstructured data sets. This approach also allows for automatically identifying new key terms related to soft skills and personality traits.

When outcome measures are available, the system may use the same foundation of key terms in predictive models based on regression, neural nets, or other approaches.

The data analytics subsystem 1300 may provide insight to commonalities and differences between job-seekers and between jobs. One key value of the personality factor determination system in the HR ecosystem is the ability to view data across many employees, teams, and job-seekers. The personality factor determination system provides insight to both organizations by mining available data for common themes across users. For example, the personality factor determination system may see that an organization has many users with high-extraversion, but not many with high conscientiousness. This information may be useful for the organization to choose which types of users to seek when hiring new employees. It can also be useful for a user to understand the personality profile of the organization to determine if they would be a good fit within the organization.

Similarly, the personality factor determination system may show how a user differs or stands out from other users. For example, suppose a user has a high personality factor score for extraversion. This may be compared to other members in a team which the user is supposed to join. In this example, the team's score for extraversion assists the organization in choosing whether to include the user within the team. Additionally, the user may be compared to the entire organization, to determine whether to include the user within the organization.

The data analytics subsystem 1300 may also provide evolving methods for determining personality profiles of users, teams, and organizations. As personality data accumulates based on the use of the personality factor determination system, the captured data may be analyzed to determine which personality traits are desirable for an organization or team. This analysis, along with qualitative feedback from users, functions to improve the personality factor determination system over time. Depending on the kind of personality data available, the personality factor determination system may use a regression-based approach or other predictive models to assess the desirability of each personality trait within the personality factor determination system.

Although embodiments described above are primarily directed to users and organizations in general, embodiments described herein can be used to assess compatibility for job-seekers and employers, personal or romantic relationships, education, and other avenues.

For example, in the relationship context, people looking for a romantic relationship are provided a plurality of attributes. These users can select content that they believe best fits the attribute or that best describes their position or believe about the attribute. Embodiments described herein can tag or label the content, which is then used to generate a personality profiles for the users. The personality profile may include personality factor scores for certain personality traits, as well as other personality traits indicated by combinations of the personality factor scores. Use of various data analytics, artificial intelligence models, or machine learning mechanisms can be used to correlate and introduce users with similar, compatible, etc., personality traits.

In the education context, prospective students can generate personality profiles, as described herein, which can be provided to universities or other educational institutions. These institutions can utilize metrics and other information from the profiles as part of the student-selection criteria, similar to an employer hiring new employees. Students can also utilize embodiments described to find funding or roommates. Typically, new students are assigned a roommate. Some institutions utilize the new student's major or hometown information to assign roommates. By employing embodiments described herein, however, new students can be matched based on their personality traits, which can result stronger bonded roommates.

FIG. 14 is a block diagram of an example processor-based device 1400 that may be used to implement at least a portion of the personality factor determination system 101 or other system discussed herein (e.g., organization system 110, candidate system 130, content system 120), according to various embodiments described herein. The processor-based device 1400 is able to read instructions from a processor-readable medium (e.g., nontransitory processor-readable storage medium) and perform any of the functionality discussed herein. The processor-based device 1400 may operate as a standalone device or may be coupled or networked to other devices. In a networked environment, the processor-based device 1400 may operate as a server device or a client device, or as a peer device in a peer-to-peer network environment. The processor-based device 1400 may comprise, for example, a server computer, a client computer, a personal computer, smartphone, a wearable computer, a tablet computer, a personal digital assistant (PDA), a laptop computer, a desktop computer, a game console, a set-top box, etc., or any of one or more devices capable of implementing the functionality described herein.

The processor-based device 1400 may include one or more processors 1402, memory 1404, and input/output (I/O) components 1406, which communicate with each other via a bus 1408. The processors 1402 may include one or more of a central processing unit (CPU), a graphics processing unit (GPU), a digital signal processor (DSP), an application specific integrated circuit (ASIC), another processor, or any suitable combination thereof. The processors 1402 may include a single processor, or a plurality of processors 1410 that execute instructions 1412. The processors 1402 may include multi-core processors that may include two or more independent processors (“cores”) that can execute instructions 1412 concurrently.

The memory 1404 may include a primary storage 1414 and a secondary storage 1416. The primary storage 1414, which may also be referred to as main memory or internal memory, may be directly accessible by the processors 1402. Non-limiting examples of the primary storage 1414 include random-access memory (RAM), read-only memory (ROM), buffer memory, flash memory, cache memory, etc. The secondary storage 1416 may include memory that is not directly accessible by the processors 1402. Non-limiting examples of the secondary storage 1416 may include solid-state memory (e.g., flash memory), optical media, magnetic media, other non-volatile memory (e.g., programmable read-only memory (PROM), or any suitable combination thereof. More generally, the primary storage 1414 and the secondary storage 1416 may include one or more nontransitory processor-readable storage media that store at least one of instructions or data that may be accessed by the processors 1402 to implement the functionality described herein. The storage 1414 and 1416 may be individual components or may include a plurality of components, and may be local, remote (e.g., cloud-based storage systems or networks), or any combination thereof.

The I/O components 1406 may include various combinations of input components 1418, output components 1420, sensors 1422, and communications components 1424, to receive input, provide output, transmit information, exchange information, capture information, etc. The I/O components 1406 may include additional or fewer components than are illustrated in FIG. 14.

The input components 1418 may include key input components (e.g., keyboard, touchscreen), point-based components (e.g., mouse, touchpad, trackball, joystick, motion sensor), tactile input components (e.g., buttons, sliders), audio input components (e.g., microphone), or other input components.

The output components 1420 may include visual components (e.g., display, projector), acoustic components (e.g., speaker), haptic components (e.g., vibrating motor), or other output components.

The sensors 1422 may include various types of sensors, including biometric sensors (e.g., gesture sensors, heart rate sensors), motion sensors (e.g., accelerometer, gyroscope), environmental sensors (e.g., illumination sensor, temperature sensor), position sensors (e.g., global positioning system (GPS) sensor), or other sensors.

The communications components 1424 may include a variety of communication technologies that operate to communicatively couple the processor-based device 1400 to a network 1426 or external devices 1428. For example, the communications components 1424 may include a network interface component or other suitable device to interface with the network 1426. The communications components 1424 may include wired communications components, wireless communications components, or combinations thereof. Non-limiting examples of wired communications include FireWire®, Universal Serial Bus® (USB), Thunderbolt®, Gigabyte Ethernet®, or any other suitable wired connection. Non-limiting examples of wireless communications include Bluetooth®, Wi-Fi®, Zigbee®, NFC (Near-field communication), cellular (e.g., 4G, 5G, etc.), RFID, or any suitable wireless connection.

The network 1426 may be any communication network or part thereof, such as an ad hoc network, the Internet, an extranet, a virtual private network (VPN), a local area network (LAN), a wide area network (WAN), a public switched telephone network (PSTN), a plain old telephone service (POTS) network, a cellular network, another type of network, or any combination of networks that allows for communication between devices.

The foregoing detailed description has set forth various implementations of the devices and/or processes via the use of block diagrams, schematics, and examples. Insofar as such block diagrams, schematics, and examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof. In one implementation, the present subject matter may be implemented via Application Specific Integrated Circuits (ASICs). However, those skilled in the art will recognize that the implementations disclosed herein, in whole or in part, can be equivalently implemented in standard integrated circuits, as one or more computer programs running on one or more computers (e.g., as one or more programs running on one or more computer systems), as one or more programs running on one or more controllers (e.g., microcontrollers) as one or more programs running on one or more processors (e.g., microprocessors), as firmware, or as virtually any combination thereof, and that designing the circuitry and/or writing the code for the software and or firmware would be well within the skill of one of ordinary skill in the art in light of this disclosure.

Those of skill in the art will recognize that many of the methods or algorithms set out herein may employ additional acts, may omit some acts, and/or may execute acts in a different order than specified.

In addition, those skilled in the art will appreciate that the mechanisms taught herein are capable of being distributed as a program product in a variety of forms, and that an illustrative implementation applies equally regardless of the particular type of signal bearing media used to actually carry out the distribution. Examples of signal bearing media include, but are not limited to, the following: recordable type media such as floppy disks, hard disk drives, CD ROMs, digital tape, and computer memory.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of operating a computer system, comprising:
determining, for at least one user-personality profile of a plurality of user-personality profiles, a personality factor score, wherein the personality factor score is determined by at least:
presenting one or more visual user interface elements to a user represented by the user-personality profile, wherein at least one of the one or more visual user interface elements comprise one or more images;
receiving an indication that the user selected at least one of the one or more visual user interface elements;
generating personality data for the user based on the selected one or more visual user interface elements; and
generating a personality factor score for the user based on the generated personality data, wherein the personality factor score represents at least one personality trait of the user.

2-3. (canceled)

4. The method of claim 1, wherein the personality data is obtained at least by:
presenting one or more words corresponding to one or more personality factors; and
obtaining an indication of which words of the one or more words were selected by the user,
wherein generating personality data for the user comprises generating personality data for the user based on the selected one or more words.

5. The method of claim 4, wherein the one or more words corresponding to the one or more personality factors are presented in connection with the one or more visual user interface elements.

6. The method of claim 1, wherein generating the personality factor score further comprises:
applying the generated personality data to a machine learning model trained to generate a personality factor score based on personality data.

7. The method of claim 6, wherein the personality factor score is generated by applying the generated personality data to the machine learning model, such that the personality factor score is generated based on a combination of imagery, machine learning, and psychometrics.

8. The method of claim 1, further comprising:
obtaining an indication of a personality trait; and
identifying one or more user-personality profiles from the plurality of user-personality profiles based on the obtained indication of a personality trait and the personality factor score of each user-personality profile of the plurality of user-personality profiles.

9. (canceled)

10. The method claim 1, wherein the personality factor score is based on at least a personality model that accounts for at least: extraversion, neuroticism, openness to experience, agreeableness, and conscientiousness.

11. The method of claim 1, further comprising:
identifying at least one user group of one or more user-personality profiles based on one or more roles within an organization; and
generating a dashboard for a user group of the at least one user group, wherein the dashboard includes an indication of the personality factor score for each of the user-personality profiles included in the user group.

12. The method of claim 1, further comprising:
identifying at least one user group of one or more user-personality profiles based on the personality factor score of each of the at least one user-personality profile.

13. The method of claim 12, further comprising:
obtaining an indication that an activity is to be generated for one user group of the at least one user group; and
generating an activity for the user group based on the personality factor score for each of the user-personality profiles included in the user group.

14. The method of claim 12, further comprising:
generating a dashboard for a user group of the at least one user group wherein the dashboard includes an indication of the personality factor score for each of the user-personality profiles included in the user group.

15. The method of claim 12, generating a group-personality profile for a user group based on the personality data of each of the user-personality profiles.

16. The method of claim 12, further comprising:
identifying correlations between each user-personality profile of the user group based on the generated personality data for each user-personality profile.

17. The method of claim 16, wherein identifying the correlations is performed by using a machine learning model that is trained to identify correlations based on personality data.

18. The method of claim 15, further comprising:
receiving an indication of a particular user; and
determining how the group-personality profile for the user group changes based on the membership of the particular user in the user group.

19. The method of claim 15, further comprising:
obtaining an indication that one or more user-personality profiles of one or more users of the user group has changed; and
altering the group-personality profile based on the indication that one or more user-personality profiles of one or more users of the user group has changed.

20. The method of claim 19, further comprising:
identifying trends in how the group-personality profile changes over time, wherein the trends include changes in morale of the group.

21. The method of claim 12, wherein identifying at least one user group is performed by using data clustering.

22. (canceled)

23. (canceled)

24. A system for determining personality factors of a user, the system comprising:
a database configured to store a plurality of digital user-personality profiles for a plurality of users;
an output interface configured to display a graphical user interface to a user; and
a processor configured to execute computer instructions to:
determine, for at least one user-personality profile of a plurality of user-personality profiles, a personality factor score, wherein the personality factor score is determined by at least:
presenting, via the graphical user interface, one or more visual user interface elements to a user represented by the user-personality profile, wherein at least one of the one or more visual user interface elements comprise one or more images;
receive, via the graphical user interface, an indication that the user selected at least one of the one or more visual user interface elements;
generate personality data for the user based on the selected one or more visual user interface elements; and
generate a personality factor score for the user based on the generated personality data, wherein the personality factor score represents at least one personality trait of the user.

25. A nontransitory processor-readable storage medium that stores computer instructions that, when executed by at least one processor, cause the at least one processor to:
determine, for at least one user-personality profile of a plurality of user-personality profiles, a personality factor score, wherein the personality factor score is determined by at least:
present one or more visual user interface elements to a user represented by the user-personality profile, wherein at least one of the one or more visual user interface elements comprise one or more images;
receive an indication that the user selected at least one of the one or more visual user interface elements;
generate personality data for the user based on the selected one or more visual user interface elements; and
generate a personality factor score for the user based on the generated personality data, wherein the personality factor score represents at least one personality trait of the user.
